(19) United States
(12) Patent Application Publication
(10) Pub. No.: US 2018/0369526 A1
(43) Pub. Date: Dec. 27, 2018

(54) Airway Suction Inlet Port

(71) Applicants: Paul A. McGowan, Kernersville, NC (US); Pamela Michelle McGowan Barker, Kernersville, NC (US)

(72) Inventors: Paul A. McGowan, Kernersville, NC (US); Pamela Michelle McGowan Barker, Kernersville, NC (US)

(21) Appl. No.: 16/017,454

(22) Filed: Jun. 25, 2018

Related U.S. Application Data
(60) Provisional application No. 62/524,904, filed on Jun. 26, 2017.

(51) Int. Cl. A61M 16/04; A61M 16/00; A61M 16/20; A61M 25/00; A61M 39/02

(52) U.S. Cl. CPC A61M 16/0463; A61M 16/0078; A61M 16/20; A61M 2025/0019; A61M 39/02; A61M 2025/0004; A61M 25/00

(57) ABSTRACT

An airway suction inlet port. The airway suction inlet port includes a housing having at least one sidewall defining an interior volume, wherein a port having a membrane thereacross is disposed on the sidewall. The port provides access to the interior volume, such that the membrane forms an airtight seal about a catheter inserted therethrough. An inlet is disposed on the housing, the inlet affixed to a bag-valve system such that air can be forced from the bag-valve system through the inlet. An outlet is disposed on the housing, the outlet capable of securing to an endotracheal tube. The catheter can then be fed through port and the outlet and into the endotracheal tube to provide suction internal to the endotracheal tube.

### CROSS REFERENCE TO RELATED APPLICATIONS

This application claims the benefit of U.S. Provisional Application No. 62/524,904 filed on Jun. 26, 2017. The above identified patent application is herein incorporated by reference in its entirety to provide continuity of disclosure.

### BACKGROUND OF THE INVENTION

The present invention relates to airway suction ports. Specifically, the present invention relates to airway inlet ports securable to a bag-valve system and an endotracheal tube, such that a user can simultaneously suction the interior of the endotracheal tube while providing oxygen to the patient.

Many patients require intubation, where an endotracheal tube is inserted into their trachea to provide an airway through which oxygen can be forced. Typically, this involves either a ventilator or a manual bag-valve system, often referred to an Ambu-bag. However, when fluid builds up within the endotracheal tube, the medical personnel must suction the fluid to provide a clear airway. If the fluid is not properly suctioned, blood and fluids can accumulate within the bag-valve system, requiring the medical professional to change to a clean bag-valve system to prevent complications. Typically, in order to provide adequate suction, the bag-valve system must be removed so that a suction tube or catheter can be inserted to provide the required suction. This can result in bodily fluids spraying onto the medical professional, as well as interrupting constant oxygen flow into the patient. Therefore, a device that allows a user to simultaneously provide suction and oxygen to a patient is desired, while preventing contamination of the surrounding area with bodily fluids.

In light of the devices disclosed in the known art, it is submitted that the present invention substantially diverges in design elements from the known art and consequently it is clear that there is a need in the art for an improvement to existing airway suction ports. In this regard, the instant invention substantially fulfills these needs.

### SUMMARY OF THE INVENTION

In view of the foregoing disadvantages inherent in the known types of airway suction ports now present in the known art, the present invention provides an airway suction port wherein the same can be utilized for providing convenience for the user when simultaneously providing suction to an interior of an endotracheal tube and providing oxygen to a patient.

The present system comprises a housing having at least one sidewall defining an interior volume, wherein a port is disposed through the sidewall, providing access to the interior volume. The port further comprises a membrane extending thereacross, wherein the membrane is configured to form a seal about a catheter inserted therethrough, such that the seal is airtight. An inlet is disposed on the housing in fluid communication with the interior volume, wherein the inlet is affixed to a bag-valve system. In an alternate embodiment, the inlet is removably securable to the bag-valve system. An outlet is disposed on the housing in fluid communication with the interior volume, wherein the outlet is configured to removably secure to an endotracheal tube. In some embodiments, the membrane comprises an aperture therein, the aperture configured to receive the catheter therethrough, and wherein the membrane is biased towards the aperture, such that the seal is formed about the catheter. In another embodiment, the outlet comprises a diameter greater than that of the endotracheal tube, such that the endotracheal tube removably secures therein via friction fit. In other embodiments, the inlet is disposed on a first end of the housing, and the outlet is disposed on the sidewall, such that the inlet extends from the housing perpendicular to the outlet. In yet another embodiment, the membrane is configured to form an airtight seal across the port when the catheter is removed therefrom.

The method for utilizing the present system comprises placing an endotracheal tube into a trachea of a patient, removably securing an inlet disposed on a housing to a bag-valve system, wherein the inlet is in fluid communication with an interior volume of the housing, removably securing an outlet disposed on the housing to the endotracheal tube, wherein the outlet is in fluid communication with the interior volume, inserting a catheter through a port disposed on a sidewall of the housing, such that the catheter enters the endotracheal tube through the interior volume such that a seal is formed about the catheter at the port via a membrane extending thereacross, and providing suction through the catheter to remove fluids from the endotracheal tube.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows an exploded view of an embodiment of the airway suction inlet port.

FIG. 2 shows a close-up view of an embodiment of the airway suction inlet port.

FIG. 3 shows a perspective view of an embodiment of the airway suction inlet port in use.

### DETAILED DESCRIPTION OF THE INVENTION

Reference is made herein to the attached drawings. Like reference numerals are used throughout the drawings to depict like or similar elements of the airway suction inlet port. The figures are intended for representative purposes only and should not be considered to be limiting in any respect.

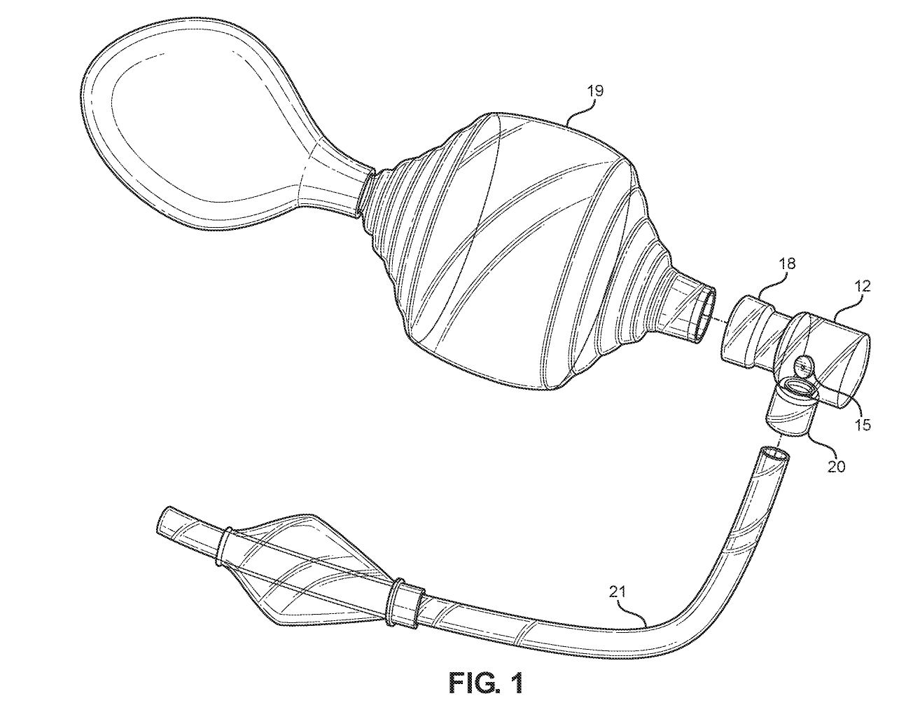

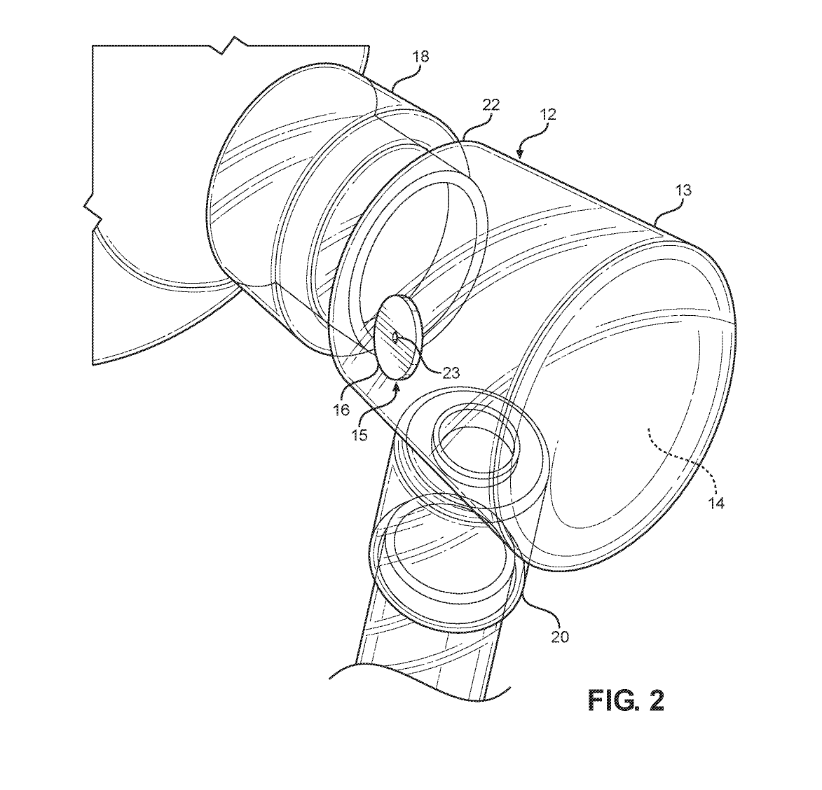

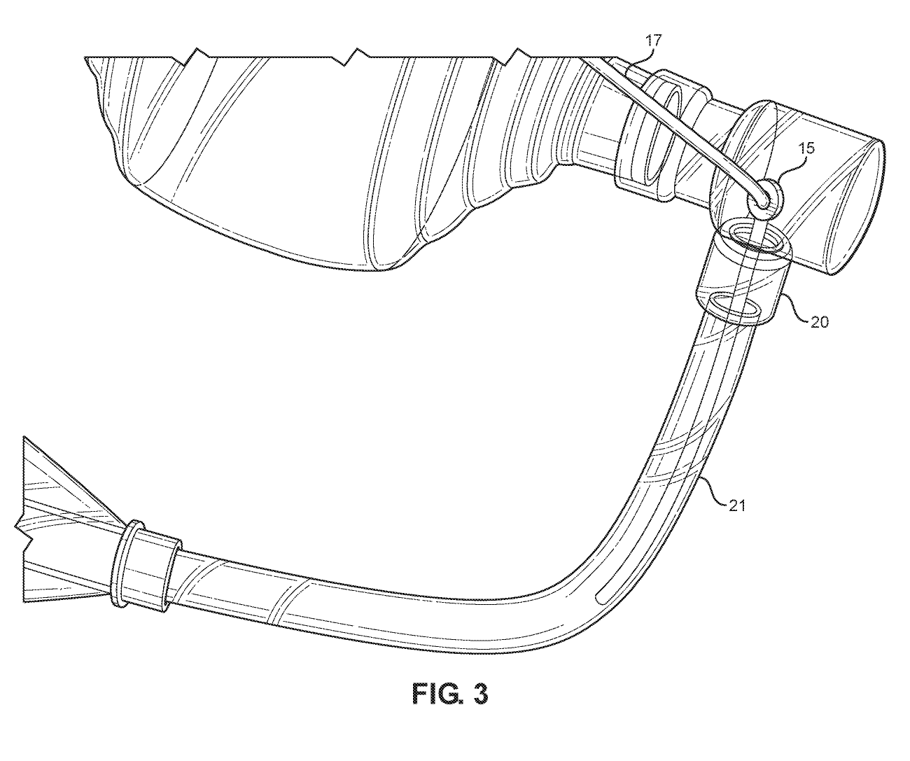

Referring now to FIG. 1, there is shown an exploded view of an embodiment of the airway suction inlet port. The airway suction inlet port comprises a housing 12 having a port 15 thereon. In the illustrated embodiment, the housing 12 comprises a cylindrical structure, however in alternate embodiments, the housing 12 comprises various alternate shapes, including, but not limited to, rectangular, spherical, or curved. The port 15 provides access to the interior volume (shown in FIG. 2, 14) of the housing 12, such that a catheter (shown in FIG. 3, 17) can be threaded through the port 15. The housing 12 provides a connection between a bag-valve system 19 and an endotracheal tube 21. In this way, the user can make use of the port 15 to suction fluids within the endotracheal tube 21 without removing the bag-valve system 19, allowing a constant stream of oxygen to a patient. In the illustrated embodiment, the housing 12 comprises a transparent material, allowing a user to easily view the interior volume to guide the catheter from the port 15 to a desired location.

In the illustrated embodiment, an inlet 18 disposed on the housing 12 is configured to removably secure a bag-valve system 19 thereto, however in alternate embodiment, the bag-valve system 19 is integral with the housing 12. In this way, the user can use existing bag-valve systems 19 with the airway suction inlet port, thereby saving the expense of replacing existing bag-valve systems 19. Alternatively, embodiments with an integral bag-valve system 19 ensure that the airway suction inlet port is always available during use, should the need to simultaneously suction fluids from the endotracheal tube 21 arise. In the illustrated embodiment, the inlet 18 comprises a diameter greater than the connector of the bag-valve system 19, such that the bag-valve system 19 is removably securable to the inlet 18 via friction fit. The bag-valve system 19 comprises a standard bag-valve system 19 having a bag configured to inflate with air, allowing a user to compress the bag to force air through the inlet 18. Typical bag-valve systems 19 comprise a connector configured to directly connect with an endotracheal tube 21, or alternatively, have a mask affixed thereto, allowing oxygen to be directed to a patient's nose and mouth.

The housing 12 further comprises an outlet 20 thereon, wherein the outlet is in fluid communication with the inlet 18, allowing fluids, such as air, to pass from the inlet 18, through the outlet 20. In the illustrated embodiment, the outlet 20 comprises a diameter greater than that of the endotracheal tube 21, such that the endotracheal tube 21 removably secures within the outlet 20 via friction fit. In this way, all airflow through the housing 12 flows into the endotracheal tube 21. Typical endotracheal tubes 21 are placed in a patient's trachea, wherein a balloon is inflated at a distal end of the endotracheal tube 21, anchoring the endotracheal tube 21 and ensuring all air flows from the endotracheal tube 21 into the patient's lungs. This also causes any bodily fluids in the patient's lungs and trachea beyond the balloon to be trapped below the balloon, such that suction must pass through the endotracheal tube 21 to effectively remove the fluids.

Referring now to FIG. 2, there is shown a close-up view of an embodiment of the airway suction inlet port. In the illustrated embodiment, the housing 12 comprises a cylindrical sidewall 13 defining an interior volume 14, however in alternate embodiments, the housing 12 can comprise additional shapes. A port 15 is disposed on the sidewall 13 providing access to the interior volume 14. In the illustrated embodiment, the port 15 is disposed adjacent to the outlet 20, allowing a user to easily thread a catheter through the port 15 and into the outlet 20, however in alternate embodiments, the port 15 can be positioned anywhere along the sidewall 13, such as directly opposite the outlet 20, allowing for greater ease of placement of the catheter therethrough.

The port 15 further comprises a membrane 16 thereacross, the membrane 16 configured to form an airtight seal about a catheter inserted therethrough. The membrane 16 can comprise various materials, such as surgical rubber, silicon, gel, and the like. In this way, air and fluids are prevented from exiting the housing 12 when the membrane 16 is pierced by the catheter. This ensures that no contamination of the surrounding area occurs during suction procedures. In the illustrated embodiment, the membrane 16 further comprises an aperture 23 therein. The aperture 23 provides an access through the membrane 16 allowing the membrane 16 to remain structurally intact, as in alternate embodiments, the membrane 16 must be pierced by the catheter. The aperture 23 is dimensioned such that a diameter of the aperture 23 is less than that of the catheter, allowing a seal to be formed therearound. In this way, the airway suction inlet port can be reused after sanitization thereof. In this embodiment, the membrane 16 is biased towards the aperture 23, such as via elastic force, such that the membrane 16 forms an airtight seal about a catheter inserted through the aperture 23, as the resting diameter of the aperture 23 comprises a diameter less than that of the catheter. Additionally, the elasticity of the membrane 16 allows the membrane 16 to form an airtight seal about the aperture 23 when no catheter is inserted therethrough, ensuring that air and fluids remain within the housing 12 during use without suction. Furthermore, in embodiments lacking the aperture 23, the membrane 16 is configured to seal over the puncture formed by the catheter insertion, such as via self-sealing gel material.

In the illustrated embodiment, the inlet 18 is disposed on a first end 22 of the housing 12 such that the inlet 18 extends in a direction perpendicular to the outlet 20. In this way, the airway suction inlet port allows the bag-valve system to be positioned horizontally over the chest of an intubated patient. This positions the bag-valves system so as to provide a comfortable grip for the user. In alternate embodiments, the inlet 18 and the outlet 20 are positioned on opposing ends of the sidewall 13, or in other orientations to allow a user to select an embodiment to provide the most comfortable position for a given situation.

Referring now to FIG. 3, there is shown a perspective view of an embodiment of the airway suction inlet port in use. In the illustrated embodiment, the catheter 17 is inserted through the port 15 such that an airtight seal is formed thereabout, ensuring that no fluids exit the housing via the port 15. The catheter 17 is threaded through the port 15 and the outlet 20 to enter the endotracheal tube 21. This allows suction applied through the catheter 17 to remove fluid accumulation within the endotracheal tube 21 while oxygen is being supplied to the user therethrough.

In one exemplary use, the user places the endotracheal tube 21 in a patient's trachea and inflates the balloon to secure the endotracheal tube 21 therein, while also ensuring that the entire volume of air delivered therethrough is directed to patient's lungs. A bag-valve system is then affixed to the inlet, and the outlet is secured to the endotracheal tube 21. The user can then deliver oxygen to the patient via pumping the bag of the bag-valve system. Should fluid buildup within the endotracheal tube 21 occur, the user can then insert a catheter 17 through the port 15 and thread the catheter 17 through the outlet 20 and into the endotracheal tube 21. The membrane within the port 15 forms an airtight seal about the catheter 17 such that any air of fluids within the housing do not exit therethrough. The user can then apply a suction force through the catheter 17 to remove fluid buildup within the endotracheal tube 21, while continuously providing oxygen to the patient via the bag-valve system.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An airway suction inlet port, comprising:
a housing having at least one sidewall defining an interior volume;
a port disposed on the sidewall providing access to the interior volume, the port having a membrane thereacross;
wherein the membrane is configured to form a seal about a catheter inserted therethrough;
wherein the seal is airtight;
an inlet disposed on the housing in fluid communication with the interior volume;
wherein the inlet is configured to removably secure to a bag-valve system;
an outlet disposed on the housing in fluid communication with the interior volume;
wherein the outlet is configured to removably secure to an endotracheal tube.

2) The airway suction inlet port of claim 1, wherein the membrane comprises an aperture therein, the aperture configured to receive the catheter therethrough, and wherein the membrane is biased towards the aperture, such that the seal is formed about the catheter.

3) The airway suction inlet port of claim 1, wherein the outlet comprises a diameter greater than that of the endotracheal tube, such that the endotracheal tube removably secures therein via friction fit.

4) The airway suction inlet port of claim 1, wherein the inlet is disposed on a first end of the housing and the outlet is disposed on the sidewall, such that the inlet extends from the housing perpendicular to the outlet.

5) The airway suction inlet port of claim 1, wherein the membrane comprises an elastic material configured to form an airtight seal across the port when the catheter is removed therefrom.

6) An airway suction inlet port, comprising:
a housing having at least one sidewall defining an interior volume;
a port disposed on the sidewall providing access to the interior volume, the port having a membrane thereacross;
wherein the membrane is configured to form a seal about a catheter inserted therethrough;
wherein the seal is airtight;
an inlet disposed on the housing in fluid communication with the interior volume;
a bag-valve system integrally affixed to the inlet;
an outlet disposed on the housing in fluid communication with the interior volume;
wherein the outlet is configured to removably secure to an endotracheal tube.

7) The airway suction inlet port of claim 6, wherein the membrane comprises an aperture therein, the aperture configured to receive the catheter therethrough, and wherein the membrane is biased towards the aperture, such that the seal is formed about the catheter.

8) The airway suction inlet port of claim 6, wherein the outlet comprises a diameter greater than that of the endotracheal tube, such that the endotracheal tube removably secures therein via friction fit.

9) The airway suction inlet port of claim 6, wherein the inlet is disposed on a first end of the housing and the outlet is disposed on the sidewall, such that the inlet extends from the housing perpendicular to the outlet.

10) The airway suction inlet port of claim 6, wherein the membrane comprises an elastic material configured to form an airtight seal across the port when the catheter is removed therefrom.

11) A method of using an airway suction inlet port, comprising:
placing an endotracheal tube into a trachea of a patient;
removably securing an inlet disposed on a housing to a bag-valve system, wherein the inlet is in fluid communication with an interior volume of the housing;
removably securing an outlet of the housing to the endotracheal tube, wherein the outlet is in fluid communication with the interior volume;
inserting a catheter through a port disposed on a sidewall of the housing, such that the catheter enters the endotracheal tube through the interior volume;
wherein a seal is formed about the catheter at the port via a membrane extending thereacross;
providing suction through the catheter to remove fluids from the endotracheal tube.
